(19) United States
(12) Patent Application Publication
(10) Pub. No.: US 2007/0259889 A1
(43) Pub. Date: Nov. 8, 2007

(54) Protein Binding Compounds

(76) Inventors: Jo Klaveness, Oslo (NO); Bjarne Brudeli, Oslo (NO)

Correspondence Address: SUGHRUE MION, PLLC, 2100 PENNSYLVANIA AVENUE, N.W., SUITE 800, WASHINGTON, DC, 20037, US

(21) Appl. No.: 10/571,890

(22) PCT Filed: Sep. 15, 2004
(86) PCT No.: PCT/GB04/03939

§ 371 Date: Feb. 15, 2007

(30) Foreign Application Priority Data
Sep. 15, 2003 (GB) 0321613.2

(51) Int. Cl. A61K 31/00; A61K 31/4164; A61K 31/513; A61K 31/52; A61K 31/7068; A61K 31/708; A61K 38/38; A61K 47/48; A61P 29/00; A61P 31/00; A61P 35/00

(52) U.S. Cl. 514/256; 514/263.3; 514/385; 514/45; 514/49; 536/27.8; 536/28.5; 544/256; 544/313; 548/327.5

(57) ABSTRACT

The present invention provides a prodrug compound comprising a therapeutically effective moiety coupled via a metabolically cleavable bond to a blood protein binding moiety.

This invention relates to protein-binding prodrug compounds, in particular compounds which are metabolized to release drug compounds effective in the treatment of cancer, inflammation, infection or pain, and to pharmaceutical compositions containing such prodrug compounds and their use in medical treatment of human or non-human animal subjects.

It is known in medical treatment to administer compounds which are therapeutically ineffective but which, in vivo, are metabolized into therapeutically effective compounds. Such therapeutically ineffective precursors are known as “prodrugs”. Equally it is known to administer therapeutically active compounds in a “camouflaged” form, e.g. encapsulated within liposomes, whereby the therapeutically active compound is not immediately available for binding to or uptake by the cells on which it is intended to act.

Many drugs administered to a vascularized animal (e.g. mammal, reptile, bird, fish, etc.) are transported to their site of action in the animal's blood. Moreover many drugs have to be administered repeatedly since a proportion of the drug molecules may be excreted or metabolized into inactive metabolites.

We have now found that drug efficacy may be enhanced and/or prolonged by the use of prodrugs which comprise a therapeutically effective moiety coupled via a metabolically cleavable bond to a blood protein binding moiety. These prodrugs are especially useful where prolonged drug action is desired, e.g. where the therapeutically effective moiety is a drug effective in the treatment of cancer, inflammation, infection, and pain, particularly cancer and pain.

Thus viewed from one aspect the invention provides a prodrug compound, preferably a water-soluble compound, comprising a therapeutically effective moiety coupled via a metabolically cleavable bond, preferably an ester bond or an oxidatively cleavable bond, especially an ester bond, to a blood protein binding moiety, preferably an acid moiety (e.g. a carboxylic acid moiety or a phosphorus oxyacid moiety, especially a carboxylic acid moiety), or an esterified acid moiety.

Viewed from a further aspect the invention provides a pharmaceutical composition, preferably a solution for injection, comprising a prodrug compound according to the invention together with at least one pharmaceutically acceptable carrier or excipient.

Viewed from a still further aspect the invention provides a method of treatment of a human or non-human vascularized animal subject, which method comprises parenterally administering to said subject (typically a subject suffering from cancer, inflammation, infection or pain) an effective amount of a prodrug according to the invention.

In the method of the invention, the prodrug compounds will typically be used to treat those conditions for which the drug moiety they contain is used to treat.

Viewed from a yet further aspect the invention provides a process for the preparation of a prodrug according to the invention which process comprises coupling (e.g. by ester formation or hydroxyl, thiol or amine alkylation) a therapeutically active drug compound (or a salt or activated derivative thereof) and a blood protein-binding agent.

The prodrug compounds according to the invention for use in medicine forms a further aspect of the invention.

By blood protein is meant herein proteins which circulate in the blood, either dissolved within the continuous aqueous phase or displayed on the surface of the blood cells. The term does not cover proteins wholly encapsulated by blood cells. Such blood proteins may or may not be glycosylated and may or may not form part of larger aggregates (e.g. as in transferrin).

Desirably the blood protein to which the prodrug may bind is one having a blood half life of at least 5 days, more preferably at least 10 days, still more preferably at least 15 days.

Examples of suitable blood proteins include transferrin, cobalamin, haptocorrin, plasma albumin, α₁ acid glycoprotein, and the cell surface proteins of erythrocytes (red blood cells). Especially preferably the blood protein is serum albumin, α₁ acid glycoprotein or an erythrocyte surface protein, most preferably it is serum albumin.

The metabolically cleavable group between the protein binding moiety and the therapeutically effective moiety is preferably an ester or an oxidatively cleavable carbon-nitrogen, carbon-sulphur or carbon-oxygen (e.g. amine, thioether or ether) bond, e.g. a bond cleavable by a CYP enzyme. Especially preferably it is an ester bond.

If desired, in the prodrugs of the invention two or more therapeutically effective moieties (the drug moieties) may be attached via metabolically cleavable bonds to a single protein binding moiety or two or more, optionally different, protein binding moieties may be attached via metabolically cleavable bonds to a single drug moiety.

The metabolically cleavable ester group in the prodrugs of the invention may be a single or multiple (e.g. double) ester group providing a —CO—O— linkage oriented in either direction (or both directions) between the protein binding moiety (V) and the active drug moiety (D). Thus for example the prodrug can take the forms:

V-(L)ₙ-CO—O-(L)ₘ-D

V-(L)ₙ-O—CO-(L)ₘ-D

V-(L)ₙ-CO—O-L-O—CO-(L)ₘ-D

V-(L)ₙ-CO—O-(L)ₚ-CO—O-(L)ₘ-D

V-(L)ₙ-O—CO-(L)ₚ-O—CO-(L)ₘ-D and

V-(L)ₙ-O—CO-(L)ₚ-CO—O-(L)ₘ-D

where n, m and p are each 0 or 1 and each L is a linker group, e.g. a C₁₋₂₀, especially C₁₋₁₀, particularly C₁₋₃, hydrocarbyl group. The linker moieties L, where present, are preferably (CH₂)_{q} groups where q is 1 to 3 or Gly and/or Cys residues or, especially preferably linker polymethylene groups interrupted by oxa groups (e.g. oligo ethyleneoxide groups) or backbone-substituted by hydrophilic groups (e.g. hydroxyl groups).

The use of double ester groups to link the drug and protein binding moiety is especially preferred.

Where the protein binding portion of the prodrug is bound via a linker to the metabolically cleavable bond, this portion may be referred to herein as a protein binding sub-unit.

Particularly desirably, the metabolically cleavable group is distanced from the protein binding sub-unit by a group —CH₂—CH₂—R— where the —CH₂—CH₂— component is attached to or by the metabolically cleavable bond (i.e. one atom may intervene between the bond and the first CH₂ group) and R is a hydrocarbyl linker containing up to 30 carbon atoms, especially 4 to 20 carbons, e.g. a linear group optionally interrupted by or terminating in a 5 to 10 membered cyclic group (for example a phenyl group). Preferably the connecting group is —(CH₂)ᵣ—R′— where r is ≧5, e.g. 9 to 22, and R′ is a bond or a hydrocarbyl linker as defined for R (less the appropriate number of carbons). Especially preferably R═(CH₂)ₛ where s≧3, preferably s=7 to 20.

The protein binding portion of the protein binding moiety in the prodrugs of the invention may be any group capable of reversibly or, less preferably, irreversibly (but not covalently) binding to a blood protein. Preferably it is one capable of binding by ionic attraction, hydrogen bonding or less preferably lipophile-lipophile attraction.

Typically such moieties will be selected from:

hydrophilic groups;

negatively charged groups, e.g. acid groups (e.g. oxyacid groups, in particular carboxylic acid and phosphorus oxyacid groups);

aromatic groups (e.g. C₅₋₁₂ groups, in particular phenyl, napthyl, etc. optionally substituted, e.g. by C₁₋₆ hydrocarbyl, cyano, or halo groups);

oligopeptides;

oligosaccharides; and

oligonucleotides.

The protein binding moiety is preferably not a non-aromatic hydrocarbyl group other than a medium to long chain non aromatic group, and especially preferably is not such a C₁₋₆ group.

Suitable protein binding groups can be identified by conventional screening techniques (e.g. phage display library scanning) and are also known from the literature. Examples of suitable groups include lectins (which can bind to glycosylated blood proteins) and RGD, or RGD analog, containing oligopeptides (see U.S. Pat. No. 5,374,622 and the publications mentioned therein and cited thereagainst).

If desired the protein binding group may itself be protected by a metabolically cleavable group, e.g. an alkyl group, especially a C₁₋₆ alkyl group e.g. a t-butyl group. Preferably this group is one which is cleaved in the gastrointestinal tract. Following administration, this protecting group is cleaved and the protein binding prodrug is formed.

Particularly preferably the residue of the prodrug of the invention which remains after metabolic cleavage of the drug moiety will be a compound which either has regulatory approval, or is rapidly excreted by glomerular filtration, or remains firmly bound to the blood protein and thus is destroyed or excreted when the protein's blood lifetime expires (e.g. where the protein binding group is a RGD- or RGD-analog-containing oligopeptide).

The protein binding moiety used in the prodrugs of the invention is one which binds reversibly, i.e. non-covalently, to a binding site on the blood protein. In this way the prodrug is in equilibrium between bound and unbound states and thus is more available for cellular uptake than would be the case where binding is irreversible, i.e. covalent. The use of RGD-like binding moieties thus preferably involves use of those moieties which bind relatively weakly.

The protein binding moiety is preferably the residue of an a ω-aromatic (e.g. phenyl or napthyl) or ω-acid (e.g. carboxylic acid) C₁₋₂₀ (especially C₁₋₁₀) linker alkanol or alkyl-carboxylic acid.

It is especially preferred that the metabolic cleavage of the prodrug of the invention is such that no more than 50%, especially no more than 20%, particularly no more than 10% is excreted uncleaved.

The drug moiety in the prodrugs of the invention is preferably released by metabolic ester cleavage as an active drug compound in carboxylic acid (or salt) or alcohol form. Particularly preferably the drug released is a compound known to the be active and having regulatory approval in such an acid (or salt) or alcohol form. However for drug compounds which do not have regulatory approval in such form, acid- or alcohol-containing analogs may be used.

The invention is particularly useful where the cleaved drug moiety (or the regulatory approved analog), when administered conventionally achieves a blood protein binding level of less than 50%, especially less than 20%, more especially less than 10%. In particular, the prodrug of the invention preferably achieves a protein binding level (i.e. percent) at least 20% higher than would the cleaved drug molecule, particularly at least 50% higher, more particularly at least 100% higher. Plasma protein binding levels and blood half-lives for many drugs can be found for example in Goodman and Gilman “The pharmacological basis of therapeutics”, 10th Edition.

The drug moiety in the prodrug of the invention is preferably an anti-cancer (e.g. cytotoxic or cytostatic) drug, or a pain relieving or suppressing drug.

Especially preferably the drug moiety is or is an analog of a drug with a blood half life of less than 5 hours, particularly less than 3 hours, e.g. in the adult human.

Examples of suitable drug compounds which may be oriented in prodrug form according to the present invention include:

azathioprine, bleomycin, busulfan, carmustine (BCNU), chorambucil, cisplatin, cyclophoaphamide, cytarabine, doxorubicin, ethanbutol, etoposide, gemcitabine, fluorocytosine, fludarbine, fluorouracil, hydroxyurea, idarubicin, ifosfamide, irinotecan, letrozole, melphalan, mercaptopurine, methotrexate, paclitaxel, thiotepa, topotean, toremifene, abacavir, acyclovir, amoxicillin, amphotericin B, ampicillin, azlocillin, carbenicillin, cefalor, cefadroxil, cefamandole, cefazolin, cefanicid, cefeprime, cefixime, cefotaxime, cefoperazone, ceforanide, cefotaxime, cefotetan, cefoxitin, cefazidime, ceftizoxime, ceftriaxone, cefuroxime, cephalexin, cephalothin, cephapirin, cepharidine, chloramphenicol, chloroquine, cinoxacin, ciprofloxacin, clarithromycin, clavulanate, clindamycin, clozacillin, dapsone, didanoside, dicloxacilline, delaviridine, doxycyclin, erythromycin, ethambutol, gentamicin, ganciclovir, gatifloxacin, imipramin, indinavir, isoniazid, itraconazole, ivermectine, kanamycin, ketoconazole, lamuvudine, mebendazole, mefloquine, methicillin, metronidazole, mezlocillin, minocycline, moxifloxacin, nelfinavir, navirapine, nitrofuratoin, ofloxacin, oseltamivir, praziquantel, quinine, quinupristin, qalfopristin, ribavirin, rifampin, rifabutin, ritonavir, saquinavir, stavudine, sulfamethoxazole, sulfasalazine, sulfisooxazole, tetracycline, thalidomide, tobramycin, trimethoprim, valacyclovir, vancomycin, zalcitabine, zanamivir, zidovudine, acetaminophen, albuterol, amikacin, atropine, cefepime, cimetidine, clonidine, codeine, ethosuximide, gabapentin, hydromorphone, isoniazid, isosorbide nitrate, levetitracetam, lisinopril, metformine, methylphenidate, metoprolol, nicotine, pacuronium, pramipexole, procainamide, ranitidine, rizatriptan, sumatriptan, tocainide, topiramate, acetylsalicyclic acid, alendronate, alfentanil, allopurinol, baclofen, benazepril, bumetamide, bupivacaine, buprenorphine, buspirone, carbidopa, carvedilol, cocaine, diclofenac, dobutamine, dolasetron, enoxaparin, entacapone, esmolol, fentanyl, fluvastatin, furosemide, gemfobrizil, glimepiride, glipzide, hydralazine, hydrochlorthiazide, ibuprofen, indomethacin, lansoprazole, levodopa, lidocaine, losartan, lovastatin, meperidine, metformine, methylprednisolone, midazolam, misoprostol, morphine, mycophenolate, nalbuphrine, naloxone, neostigmine, nicardipine, nifedipine, nitrofurantoin, nitroglycerin, omeprazole, ondasetron, oxcarbazepine, oxybutyrin, oxytocin, phenylephrine, pravastatin, prazosin, prednisolone, prednisone, propofol, propranolol, rapacuronium, remifentanil, repaglinide, risperidone, rosiglitazone, selegeline, sibutramine, sildenafil, simvastatin, sufentanil, sulindac, tolcapone, tolterodine, triazolam, zaleplon, zileuton, zolmitriptan and zolpidem.

Especially preferred drug compounds are:

metronidazole, 6-mercaptopurine, 5-fluorouracil, cytarabine (ara-C) and didanosine (ddI, dideoxyinosine).

Therefore, viewed from a further aspect, the invention provides a prodrug compound comprising a therapeutically effective moiety coupled via a metabolically cleavable bond to a blood protein binding moiety, wherein the therapeutically effective moiety is selected from the group consisting of metronidazole, 6-mercaptopurine, 5-fluorouracil, cytarabine and didanosine.

Preferably the protein binding moiety is an acid, e.g. a carboxylic acid or a phosphorus oxyacid, especially preferably it is an ester-bound azelaic acid, optionally with its second carboxyl group ester-protected.

Thus viewed from a further aspect the present invention provides a prodrug compound comprising a therapeutically effective moiety coupled via a metabolically cleavable bond to a blood protein binding moiety, wherein the therapeutically effective moiety is selected from the group consisting of metronidazole, 6-mercaptopurine, 5-fluorouracil, cytarabine and didanosine and the protein binding moiety is an ester-bound azelaic acid, optionally with its second carboxyl group ester-protected.

The metabolically cleavable group is preferably distanced from the protein binding moiety by a group —CH₂—CH₂—R— as previously defined, more preferably R═(CH₂)ₛ where s≧3, preferably s=7 to 20.

The preferences for the metabolically cleavable moiety expressed herein may be applied both to drug moieties as a whole and to the individually named drug moieties. However, in the case of metronidazole, the cleavable moiety preferably comprises a group (CH₂)ₛR′ (where s≧7) and/or the protein binding sub-unit is preferably a phosphorus oxyacid (or ester); in the case of cytarabine the cleavable moiety preferably comprises a group (CH₂)ₛR′ (where s>3) and/or an optionally substituted phenylalkylcarbonyl group; in the case of 5-fluorouracil, the cleavable moiety preferably comprises a group (CH₂)ₛR′ (where s≧3) and/or the protein binding sub-unit is preferably a phosphorus oxyacid (or ester); and in the case of didanosine the cleavable moiety preferably comprises a group (CH₂)ₛR′ (where s≧4) and/or an optionally substituted phenylalkylcarbonyl group and/or the protein binding sub-unit is preferably a phosphorus oxyacid (or ester).

Such prodrug compounds are suitable for use in therapy and/or as a medicament.

Thus, from a further aspect, the invention provides a prodrug compound comprising a therapeutically effective moiety coupled via a metabolically cleavable bond to a blood protein binding moiety, wherein the therapeutically effective moiety is selected from the group consisting of metronidazole, 6-mercaptopurine, 5-fluorouracil, cytarabine and didanosine, for use as a medicament.

The prodrugs of the invention may be prepared by conventional synthetic techniques for ester formation by reacting a protein binder precursor with a drug precursor optionally simultaneously or subsequent to reaction with a bifunctional linker moiety.

As mentioned above, the prodrugs of the invention are preferably water-soluble, e.g. at least 1 mg in 1000 mL at 21° C., especially at least 1 mg in 100 mL, more especially at least 1 mg in 30 mL.

The prodrug of the invention may be formulated with conventional pharmaceutical carriers or excipients (e.g. solvents, pH modifiers, viscosity modifiers, stabilizers, chelators, etc.) and in conventional administration forms (e.g. solutions, powders, dispersions, etc.).

The prodrugs of the invention will typically be administered at dosage levels below or comparable to those conventional for the cleaved drug moiety, e.g. at 5 to 100% of the conventional dosage, more especially 10 to 80%.

Viewed from yet a further aspect the invention provides a method of producing a parenteral pharmaceutical composition, said method comprising selecting a drug compound which has regulatory approval in the EU or the US in a non-ester alcohol, acid or acid salt form; manufacturing an ester of said drug compound; formulating said ester into a parenterally administrable form together with a physiologically acceptable carrier or excipient (e.g. water for injections); and sterilizing and packaging said form.

The invention will now be illustrated further by the following non-limiting Examples:

### EXAMPLE 1

### 5′-Gemcitabine mono ester with azelaic acid

2′2′-Difluorodeoxyribofuranosyl cytosine (Gemcitabine) (0.26 g, 1 mmol) is dissolved in DMF (20 ml). HCl gas is added (1 mmol). A solution of crude azelaic acid mono acid chloride (prepared from azelaic acid and thionyl chloride) (0.23 g, 1.1 mmol) in DMF (3 ml) is added and the mixture is stirred for 24 hours. The solvent is evaporated at high vacuum and the crude product is purified on a column of silica gel. The title compound is isolated.

### EXAMPLE 2

### 5′(2′3′-dideoxy-cytidine) mono ester with 1,12-dodecanedicarboxylic acid

The title compound is prepared from 2′,3′-dideoxy-cytidine and 1,12-dodecanedicarboxylic acid according to method described in Example 1.

### EXAMPLE 3

### Sebacic Acid Monomethyl Ester Chloride

The acid chloride is made from sebacic acid monomethyl ester and thionyl chloride according to Organic Synthesis Coll Vol 3, p 613.

### EXAMPLE 4

### 2-Amino-1,9-dihydro-9-(2′-(1-(10-acetyl-decanoyloxy)ethoxymethyl))-guanine

(Unsymmetrical diester of sebacic acid with methanol and 2′-acyclovir) (Prodrug)

Acyclovir (0.23 g, 1 mmol) is dissolved in dry pyridine (10 ml) and DMF (5 ml). A solution of the acid chloride from Example 3 (0.31 g, 1.3 mmol) in dichloromethane (3 ml) is added. The mixture is stirred at ambient temperature until TLC shows that no or very little acyclovir is left. The mixture is evaporated and the title compound is isolated after flash chromatography on silica.

### EXAMPLE 5

### 5′-Cytarabine monoester with 1,4-phenylene diacetic acid

The title compound is prepared from cytarabine and 1,4-phenylene diacetic acid according to method described in Example 1.

### EXAMPLE 6

### Powder for Injection

The ester from Example 5 (200 mg) is dissolved in water for injections (50 ml) by adding one equivalent of sodium hydroxide. The mixture is filtered through a 0.22 micrometer filter and filled into a 50 ml vial. The vial is freeze dried leaving the cytarabine monoester as sodium salt.

The powder is dissolved in water for injections before administration intravenously.

### EXAMPLE 7

### Protein Binding Studies

The compound (approximately 5 mg) was dissolved in DMSO (approximately 1.8 ml). A small sample of this DMSO solution (approximately 5 mg) was added to a solution of bovine serum albumin (BSA) in water (approximately 1.8 ml, 4% BSA). Another sample of the DMSO solution (approximately 5 mg) was added to a solution of phosphate buffer pH 7.4 (isotonic) (approximately 1.8 ml).

Both samples were shaken in a water bath for 30 minutes at 37° C.

The samples were transferred to centrifugal filter devices (Millipore Centricom YM-10, regenerated cellulose 10,000 MWCO. Cat. No. 4203). The devices were centrifugated at 4000 rpm for 1 hour at 37° C.

The amount of drug in the filtrates was determined by HPLC(C-18 column, phosphate buffer pH 2.2/acetonitrile, UV detection).

The phosphate buffer sample serves as a reference sample with no protein binding.

### EXAMPLE 8

### Synthesis of 4-(1H-purin-6-ylthio)pentanoic acid ethyl ester

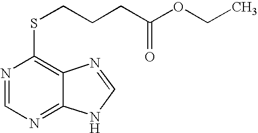

6-Mercaptopurine hydrate (510 mg, 3.0 mmol) and KOH (198 mg, 3.0 mmol) in MeOH (5 ml) was added to a suspension of ethyl 4-bromobutyrate (585 mg, 3.0 mmol) and NaI (450 mg, 3.0 mmol) in acetone (5 ml) and the mixture stirred at room temperature for 12 h. The reaction mixture was filtered and the filtrate evaporated in vacuo. The residue was added to Et₂O and a white precipitate formed. The precipitate was transferred to a flash column with silica gel and separated with CH₂Cl₂/MeOH (7:1) as eluent system to leave the product as a white crystalline solid. Yield: 568 mg (71.2%)

¹H-NMR (DMSO-d₆, 200 MHz): δ 13.53 (br s, 1H), 8.69 (s, 1H), 8.45 (s, 1H), 4.12-4.02 (q, 2H), 3.43-3.35 (m, 2H), 2.51-2.43 (m, 2H), 2.06-1.92 (m, 2H), 1.18 (t, 3H).

### EXAMPLE 9

### Synthesis of 6-(1H-purin-6-ylthio)-hexanoic acid methyl ester

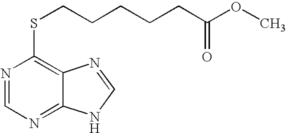

6-Mercaptopurine hydrate (510 mg, 3.0 mmol) and KOH (198 mg, 3.0 mmol) in MeOH (5 ml) was added to a suspension of methyl 6-bromohexanoate (630 mg, 3.0 mmol) and NaI (450 mg, 3.0 mmol) in acetone (5 ml) and the mixture stirred at room temperature for 12 h. The reaction mixture was filtered and the filtrate evaporated in vacuo. The residue was added to Et₂O and a white precipitate formed. The precipitate was transferred to a flash column with silica gel and separated with CH₂Cl₂/MeOH (7:1) as eluent system to leave the product as a white crystalline solid. Yield: 525 mg (61.9%)

¹H-NMR (CDCl₃, 200 MHz): δ 8.73 (s, 1H), 8.27 (s, 1H), 3.64 (s, 3H) 3.37 (t, 2H), 2.31 (t, 2H), 1.86-1.75 (m, 2H), 1.71-1.60 (m, 2H), 1.56-1.42 (m, 2H).

### EXAMPLE 10

### Synthesis of 12-(1H-purin-6-ylthio)-dodecanoic acid methyl ester

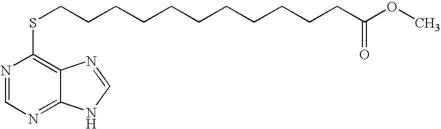

6-Mercaptopurine hydrate (611 mg, 3.6 mmol) and KOH (237 mg, 3.6 mmol) in MeOH (5 ml) was added to a suspension of methyl 12-bromododecanoate (1.05 mg, 3.6 mmol) and NaI (539 mg, 3.6 mmol) in acetone (5 ml) and the mixture stirred at room temperature for 12 h. The reaction mixture was filtered and the filtrate evaporated in vacuo. The residue was added to Et₂O and a white precipitate formed. The precipitate was transferred to a flash column with silica gel and separated with CH₂Cl₂/MeOH (7:1) as eluent system to leave the product as a white crystalline solid. Yield: 606 mg (46.2%).

¹H-NMR (DMSO-d₆, 300 MHz): δ 8.62 (s, 1H), 8.35 (s, 1H), 3.55 (s, 3H), 3.31 (t, 2H), 2.25 (t, 2H), 1.72-1.63 (m, 2H), 1.47-1.39 (m, 4H), 1.21 (br s, 12H).

¹³C-NMR (DMSO-d₆, 75 MHz): δ 173.3, 157.5, 151.1, 151.0, 144.2, 129.6, 51.1, 33.2, 29.15, 28.8, 28.7, 28.6, 28.5, 28.4, 28.1, 27.7, 24.3.

### EXAMPLE 11

### Synthesis of 12-(1H-purin-6-ylthio) dodecanoic acid

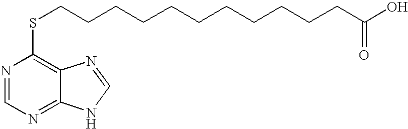

To 12-(1H-purin-6-ylthio) dodecanoic acid methyl ester (364 mg, 1.0 mmol) in H₂O (5 ml) was added KOH (145 mg, 2.2 mmol) and stirred at room temperature for 12 h, filtered and the filtrate cooled to °0 C. on an ice bath. 1 M aqueous HCl was added dropwise to the solution to leave the free acid as a white precipitate. Yield: 274 mg (78.4%).

¹H-NMR (DMSO-d₆): δ 8.86 (s, 1H), 8.24 (s, 1H), 3.30 (t, 2H), 2.12 (t, 2H), 1.71-1.60 (m, 2H), 1.41-1.21 (m, 16H).

### EXAMPLE 12

### Synthesis of 5-fluoro-1-9(9-oxononanoic acid)2,4[1H,3H]-pyrimidinedione

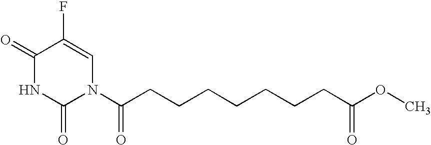

To 5-Fluorouracil (390 mg, 3.0 mmol) and NEt₃ (303 mg, 3.0 mmol) in DMF (5 ml) was added the acid chloride of monomethyl azelaic acid (728 mg, 3.3 mmol) in DMF (2 ml) and stirred at room temperature overnight. The reaction mixture was filtered and the filtrate evaporated in vacuo. The residue was transferred to a flash column with silica gel and separated with CH₂Cl₂/MeOH (10:1) as eluent system to leave the expected product as a white solid. Yield: 372 mg, (39.5%).

¹H-NMR (DMSO-d₆, 300 MHz): δ 8.83 (br s, 1H), 8.26 (d, 1H), 3.64 (s, 3H), 3.09 (t, 2H), 2.28 (t, 2H), 1.72-1.56 (m, 4H), 1.38-1.22 (m, 6H).

¹³C-NMR (CDCl₃, 75 MHz): δ 174.2, 171.8, 156.6, 156.2, 147.6, 139.6, 121.9, 121.4, 51.4, 38.9, 33.9, 28.8, 28.6, 24.7, 24.2.

### EXAMPLE 13

### Synthesis of 5-Fluoro-2,4-dioxo-1,3 (2H,4H)-pyrimidine didodenanoic acid dimethyl ester

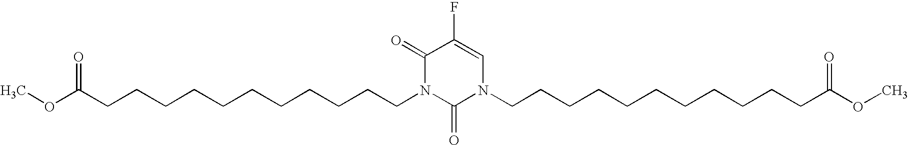

To 5-Fluorouracil (390 mg, 3.0 mmol) in DMF (5 ml) was added anhydrous K₂CO₃ (829 mg, 6 mmol) and stirred at room temperature overnight. To the 5-FU potassium salt was added methyl 12-bromododecanoate (1.76 g, 6.0 mmol) in DMF (5 ml) and stirred for 12 h. The reaction mixture was filtered and the filtrate was evaporated in vacuo. The residue was transferred to a flash column with silica gel and separated with CH₂Cl₂/MeOH (10:1) as eluent system to leave the expected product as a white solid. Yield: 258 mg, (15.4%).

¹H-NMR (DMSO-d₆, 200 MHz): δ 7.19 (d, 1H), 3.98-3.90 (m, 2H), 3.74-3.68 (m, 8H), 2.30 (t, 4H), 1.64-1.57 (m, 8H), 1.26 (br s, 28H).

### EXAMPLE 14

### Synthesis of Hexadecandioic acid, mono[2-(2-methyl-5-nitro-1H-imidazol-1-yl)ethyl]ester methyl ester

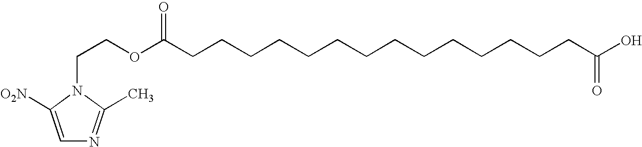

To hexadecanedioic acid (1.14 g, 4.0 mmol) in THF (40 ml) was added DCC (412 mg, 2.0 mmol) and stirred at room temperature for 2 h. The reaction mixture was added metronidazol (340 mg, 2.0 mmol) and DMAP (122 mg, 1.0 mmol) and the reaction mixture stirred overnight. The mixture was filtered and the filtrate was evaporated in vacuo. The residue was transferred to a flash column with silica gel and separated with CH₂Cl₂/MeOH (10:1) as eluent to leave the expected product as a white solid. Yield: 327 mg, (37.2%).

¹H-NMR (DMSO-d₆, 200 MHz): δ 11.96 (s, 1H), 8.04 (s, 1H), 4.60 (t, 2H), 4.38 (t, 2H), 3.34 (s, 1H), 2.47 (s, 3H), 2.26-2.16 (m, 4H), 1.50-1.40 (m, 4H), 1.25 (br s, 20H).

### EXAMPLE 15

### Synthesis of 1,2-Benzenedicarboxylic acid, mono[2(2-methyl-5-nitro-1H-imidazol-1-yl)ethyl]ester

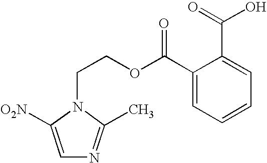

To metronidazol (1.71 g, 10.0 mmol) in CH₃CN (150 ml) was added phthalic anhydride (1.48 g, 10.0 mmol) and DMAP (122 mg, 1.0 mmol) and stirred at room temperature overnight. The title compound was collected as a precipitate, filtered and dried. Yield: 2.78 g (87.1%)

¹H-NMR (DMSO-d₆): δ 13.3 (br s, 1H), 8.06 (s, 1H), 7.82-7.68 (m, 1H), 7.66-7.61 (m, 2H), 7.56-7.48 (m, 1H), 4.71-4.56 (m, 4H), 2.45 (s, 3H)

### EXAMPLE 16

### Synthesis of 1-(5-O-Azelayl-β-D-arabinofuranosyl)cytosine methyl ester

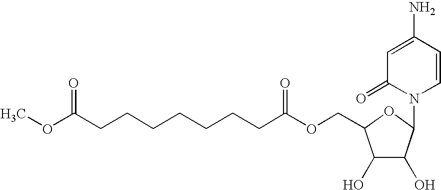

A suspension of cytarabin hydrochloric salt (prepared from cytarabin and 1M HCl in diethyl ether) (839 mg, 3.0 mmol) in DMA (10 ml) was added to the acid chloride of monomethyl azelaic acid (761 mg, 3.45 mmol) in DMA (2 ml) and the mixture stirred at room temperature overnight. NEt₃ was added to the reaction mixture (303 mg, 3.0 mmol) and the mixture evaporated in vacuo. The residue was transferred to a flash column with silica gel and separated with (CH₂Cl₂/MeOH 10:1) as eluent system to leave the product as a white solid. Yield: 278 mg, (21.6%).

¹H-NMR (DMSO-d₆,300 MHz): δ 7.47 (d, 1H), 7.10 (br d, 2H), 6.09 (d, 1H), 5.67 (d, 1H), 5.60-5.56 (m, 2H), 4.33-3.88 (m, 2H), 3.98-3.96 (m, 1H), 3.94-3.88 (m, 2H), 3.57 (s, 3H), 3.33 (br s, 1H), 2.34-2.25 (m, 4H), 1.54-1.47 (m, 4H), 1.22 (br s, 6H).

¹³C-NMR (DMSO-d₆, 75 MHz): δ 173.3, 172.8, 165.5, 155.0, 142.8, 92.5, 86.1, 81.7, 76.7, 74.2, 63.6, 51.1, 45.2, 33.5, 33.1, 28.2, 28.1, 24.3, 8.4.

### EXAMPLE 17

### Synthesis of cytarabin-N-acyl ester derivative (10a)

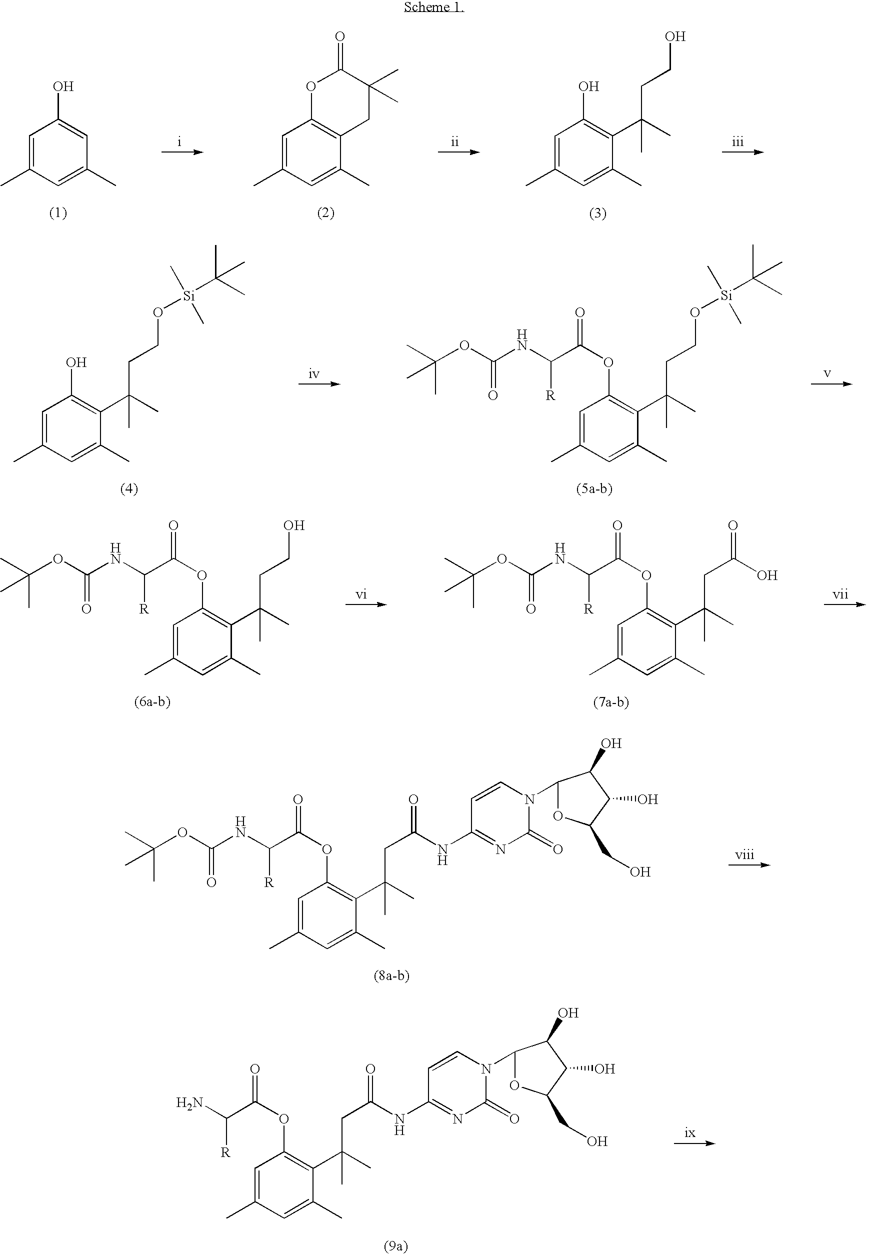

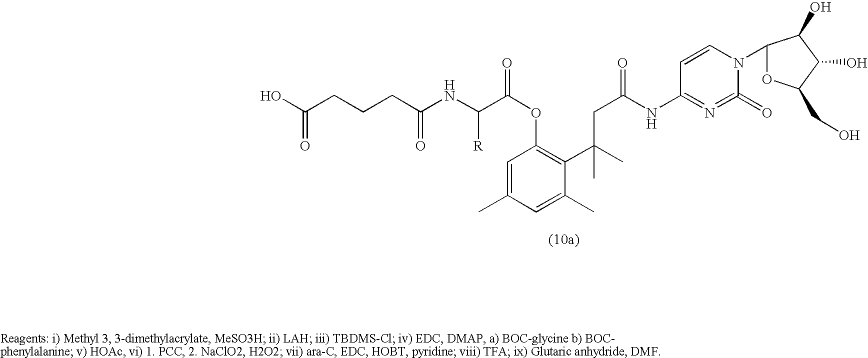

The compounds 1-4 were prepared according to Greenwald, R. B., Choe, Y. H., Conover, C. D., Shum, K., Wu, D., Royzen, M. J. Med. Chem., 2000, 43, 475-487.

### EXAMPLE 17.1

### Synthesis of 1-O-(tert-Butyldimethylsilyl)-3-(2′-BOC-glycinyl-4′,6′-dimethylphenyl)-3,3-dimethylpropanol (5a)

EDC.HCl (1.75 g, 9.3 mmol) was added to a mixture of 4 (1.00 g, 3.1 mmol), BOC-Gly-OH (1.09 g, 6.2 mmol) and DMAP (2.65 g, 21.7 mmol) in anhydrous CH₂Cl₂ (10 ml) at 0° C. The mixture was stirred overnight and 100 ml CH₂Cl₂ was added. The solution washed with 1% NaHCO₃ (3×50 ml), 1 M HCl (3×50 ml) and dried over MgSO₄. The solvent was removed in vacuo and the residue was purified by flash chromatography (3% MeOH in CH₂Cl₂). Yield: 1.2 g.

¹H NMR (CDCl₃) δ 6.84 (d, 2.5 Hz, 1H), 6.59 (s, 1H), 5.19 (s, broad, 1H), 4.15 (d, 5.5 Hz, 2H), 3.50 (t, 7.6 Hz, 2H), 2.54 (s, 3H), 2.24 (s, 3H), 2.02 (t, 7.5 Hz, 2H), 1.48 (d, 6+9H), 0.86 (s, 9H), 0.00 (s, 6H);

¹³C NMR δ 170.03, 156.07, 149.72, 138.96, 136.52, 134.41, 133.01, 123.16, 80.49, 61.12, 53.82, 46.43, 43.71, 39.49, 32.18, 28.72, 26.33, 25.61, 20.54, 18.61, −4.94.

### EXAMPLE 17.2

### Synthesis of 1-O-(tert-Butyldimethylsilyl)-3-(2′-BOC-phenylalaninyl-4′,6′-dimethylphenyl)-3,3-dimethylpropanol (5b)

Prepared by reacting EDC.HCl (1.92 g, 10 mmol), 4 (1.61 g, 5 mmol), BOC-Phe-OH (1.99 g, 7.5 mmol) and DMAP (3.05 g, 25 mmol) in anhydrous CH₂Cl₂ (75 ml) as described for 5a. Yield: 2.6 g (90%).

¹H NMR (CDCl₃) δ 7.29 (m, 5H), 6.78 (s, 1H), 6.26 (s, 1H), 5.09 (d, 1H), 4.75 (q, 1H), 3.50 (t, 7.5 Hz, 2H), 3.21 (m, 2H), 2.55 (s, 3H), 2.23 (s, 3H), 2.08 (m, 2H), 1.49 (d, 6+9H), 0.87 (s, 9H), 0.00 (s, 6H).

### EXAMPLE 17.3

### Synthesis of 3-(2′-BOC-Glycinyl-4′,6′-dimethylphenyl)-3,3-dimethylpropanol (6a)

A mixture of 5a (1.2 g, x mmol), THF (5 ml), H₂O (5 ml) and HOAc (15 ml) was stirred at room temperature for 1 h. The solvent was removed in vacuo to give the product as an oil. The product was used without further purification.

### EXAMPLE 17.4

### Synthesis of 3-(2′-BOC-Phenylalaninyl-4′,6′-dimethylphenyl)-3,3-dimethylpropanol (6b)

A mixture of 5b (2.6 g), THF (10 ml), H₂O (10 ml) and HOAc (30 ml) was stirred at room temperature for 1 h. The solvent was removed in vacuo to give the product as an oil. The product was used without further purification.

### EXAMPLE 17.5

### Synthesis of 3-(2′-BOC-Glycinyl-4′,6′-dimethylphenyl)-3,3-dimethylpropionic acid (7a)

A solution of 6a (1.04 g, 2.85 mmol) in anhydrous CH₂Cl₂ (20 ml) was slowly added to a suspension of PCC (1.35 g, 6.25 mmol) of anhydrous CH₂Cl₂ (50 ml) and stirred overnight at room temperature. The reaction mixture was evaporated in vacuo and the residue was dissolved in CH₂Cl₂ (30 ml) followed by filtration through a plug of silica (5 cm). The silica gel was flushed with diethyl ether 5 times. The filtrate was evaporated to give the a viscous oil. The oil was dissolved CH₃CN (ml) and slowly added to a solution of NaH₂PO₄ in H₂O, followed by slowly addition of 80% sodium chlorite (0.68 g, 7.5 mmol) in H₂O (12 ml) at 0° C. The reaction mixture was stirred for 1 h at 0° C. and then allowed to reach room temperature. Sodium sulfite was added to the reaction to decompose HOCl and H₂O₂. The pH was adjusted to 2 using 1M aqueous HCl, followed by extraction with ethyl acetate. The organic phase washed with brine and dried over anhydrous MgSO₄. The solvent was removed in vacuo and the residue was purified by flash chromatography (10-20% MeOH in CH₂Cl₂) to give the product. Yield: 0.65 g.

¹H NMR (CDCl₃) δ 6.84 (d, 1.5 Hz, 1H), 6.62 (s, 1H), 5.28 (s, broad, 1H), 4.15 (d, 5.5 Hz, 2H), 2.82 (s, 2H), 2.55 (s, 3H), 2.24 (s, 3H), 1.58 (s, 6H), 1.48 (s, 9H).

### EXAMPLE 17.6

### Synthesis of 3-(2′-BOC-Phenylalaninyl-4′,6′-dimethylphenyl)-3,3-dimethylpropionic acid (7b)

A solution of 6b (2.64 g, 4.9 mmol) in anhydrous CH₂Cl₂ (50 ml) was slowly added to a suspension of PCC (2.33 g, 10.8 mmol) of anhydrous CH₂Cl₂ (100 ml) and stirred overnight at room temperature. The reaction mixture was evaporated in vacuo and the residue was dissolved in CH₂Cl₂ (30 ml) followed by filtration through a plug of silica (5 cm). The silica gel was flushed with diethyl ether 5 times. The filtrate was evaporated to give the a viscous oil. The oil was dissolved in CH₃CN (9 ml) and added to a solution of NaH₂PO₄ (0.25 g, 1.8 mmol) in H₂O (4 ml), followed by slowly addition of 80% sodium chlorite (0.68 g, 7.5 mmol) in H₂O (12 ml) at 0° C. The reaction mixture was stirred for 1 h at 0° C. and then allowed to reach room temperature. Sodium sulfite (0.38 g, 3 mmol) was added to the reaction to decompose HOCl and H₂O₂. The pH was adjusted to 2 using 1M aqueous HCl, followed by extraction with ethyl acetate. The organic phase washed with brine and dried over anhydrous MgSO₄. The solvent was removed in vacuo and the residue was purified by flash chromatography (10-20% MeOH in CH₂Cl₂) to give the product. Yield: 1.2 g.

¹H NMR (CDCl₃) δ 9.03 (s, broad, 1H), 7.21 (m, 5H), 6.65 (s, 1H), 6.18 (s, 1H), 5.04 (d, Hz, 1H), 4.65 (q, Hz, 1H), 3.30-2.97 (m, 2H), 2.75 (m, 2H), 2.41 (s, 3H), 2.10 (s, 3H), 1.60 (s, 3H), 1.45 (s, 3H), 1.36 (s, 9H);

¹³C NMR δ 176.38, 171.76, 155.88, 149.73, 138.63, 136.69, 136.20, 133.90, 133.07, 129.96, 129.12, 127.66, 122.86, 80.89, 55.45, 47.98 39.12, 38.16, 31.86, 31.69, 28.67, 25.76, 20.63.

### EXAMPLE 17.7

Synthesis of Cytarabin Derivative (8a).

A Mixture of 7a (0.50 g, 1.3 mmol), ara-C (1.26 g, 5.2 mmol), HOBT (0.70 g, 5.2 mmol) and EDC.HCl (1.99 g, 10.4 mmol) in anhydrous pyridine (30 ml) was stirred at room temperature for 2 h followed by stirring at 40° C. overnight. The solvent was removed in vacuo and the residue was dissolved in CH₂Cl₂ (50 ml) followed by extraction with water (3×30 ml) and 0.1 M HCl (2×30 ml). The organic phase was dried over anhydrous MgSO₄ and evaporated in vacuo. The residue was purified by flash chromatography (5-10% MeOH in CH₂Cl₂) to give white crystalline material. Yield: 0.29 g (36%).

¹H NMR (CDCl₃) δ 9.70 (d, broad, 1H), 8.08 (s, 1H), 7.28 (q, 1H), 6.79 (s, 1H), 6.59 (s, 1H), 6.10 (s, 1H), 5.83 (s, 1H), 5.55-5.39 (m, 2H), 4.71 (s, 1H), 4.47 (s, 1H), 4.25 (s, 1H), 4.07 (d, 3H), 3.81 (s, broad, 2H), 3.01-2.77 (m, 2H), 2.49 (s, 3H), 2.18 (s, 3H), 1.56-1.43 (m, 6+9H).

### EXAMPLE 17.8

Synthesis of Cytarabin Derivative (8b).

A mixture of 7b (0.67 g, 1.4 mmol), ara-C (1.36 g, 5.6 mmol), HOBT (0.76 g, 5.6 mmol) and EDC.HCl (2.15 g, 11.2 mmol) in anhydrous pyridine (35 ml) was stirred at room temperature for 2 h followed by stirring at 40° C. overnight. The solvent was removed in vacuo and the residue was dissolved in CH₂Cl₂ (50 ml) followed by extraction with water (3×30 ml) and 0.1 M HCl (2×30 ml). The organic phase was dried over anhydrous MgSO₄ and evaporated in vacuo. The residue was purified by flash chromatography (5-10% MeOH in CH₂Cl₂) to give white crystalline material. Yield: 0.35 g.

¹H NMR (CDCl₃) δ 9.35-9.01 (m, 1H), 8.59 (m, 1H), 8.15 (m, 1H), 7.69 (m, 1H), 7.28 (m, 6H), 6.75 (d, 1H), 6.23 (t, 1H), 6.16 (t, 1H), 5.90-5.74 (m, 1H), 5.25 (s, 1H), 4.84 (m, 1H), 4.59 (s, 1H), 4.33 (s, 1H), 4.15 (s, 1H), 3.92-3.67 (m, 2H), 3.35-3.25 (m, 1H), 3.20-3.12 (m, 1H), 2.88 (s, 2H), 2.54-2.41 (m, 3H), 2.23 (d, 3H), 1.58-1.44 (m, 6H), 1.38 (s, 9H);

¹³C NMR δ 173.59, 172.30, 162.45, 156.40, 156.03, 150.23, 149.99, 147.04, 138.60, 137.13, 136.65, 136.42, 133.71, 133.31, 129.92, 129.70, 129.01, 127.52, 124.28, 123.02, 96.95, 89.22, 86.50, 80.63, 74.77, 62.36, 55.53, 50.71, 40.02, 38.33, 32.57, 31.93, 28.71, 25.82, 20.56.

### EXAMPLE 17.9

Synthesis of Cytarabin Derivative (9a).

TFA (10 ml) was added to a solution of 8a (0.29 g) in CH₂Cl₂ (10 ml) and the mixture was stirred for 2 h. The solvent was evaporated in vacuo and diethyl ether was added to precipitate the product as the TFA-salt.

### EXAMPLE 17.10

### Synthesis of HO₂C(CH₂)₃CO-Phe-TML-cytarabin (10a)

Glutaric anhydride (0.042 g, 0.56 mmol) was added to a solution of 9a (0.228 g, 0.37 mmol) and triethylamine (0.06 g, 0.006 mmol) in DMF (10 ml) at 0° C. The reaction mixture was stirred overnight and evaporated in vacuo. The residue was dissolved in MeOH (0.5 ml) and the product was precipitated by adding diethyl ether. The white crystalline material was collected by filtration. Yield: 0.15 g.

¹H NMR (CD₃OD-CDCl₃) δ 8.16 (d, 7.3 Hz, 1H), 7.33 (d, 7.4 Hz, 1H), 6.83 (s, 1H), 6.59 (s, 1H), 6.20 (s, 1H), 4.26 (s, 2H), 4.17 (d, 3H), 4.07 (s, 1H), 3.84 (q, 2H), 3.50 (q, 1H), 3.34 (s, 1H), 3.16 (q, 1H), 2.94 (t, 2H), 2.56 (s, 3H), 2.38 (d, 5H), 2.21 (s, 3H), 1.96 (t, 2H), 1.62 (s, 6H), 1.31 (t, 7.0 Hz, 2H), 1.20 (t, 7.0 Hz, 2H);

¹³C NMR δ 180.22, 179.10, 176.47, 174.07, 166.96, 160.64, 153.64, 150.63, 142.56, 140.72, 137.78, 136.95, 126.95, 100.47, 92.47, 90.15, 81.20, 79.30, 70.16, 65.78, 54.28, 51.89, 46.40, 43.45, 38.84, 37.29, 35.75, 35.66, 29.46, 25.05, 24.03, 18.98, 12.64.

Cytarabin derivative (10b) can be prepared from compound 8b similarly to compound (10a).

### EXAMPLE 18

### Synthesis of cytarabin N-acyl-ester derivative (17)

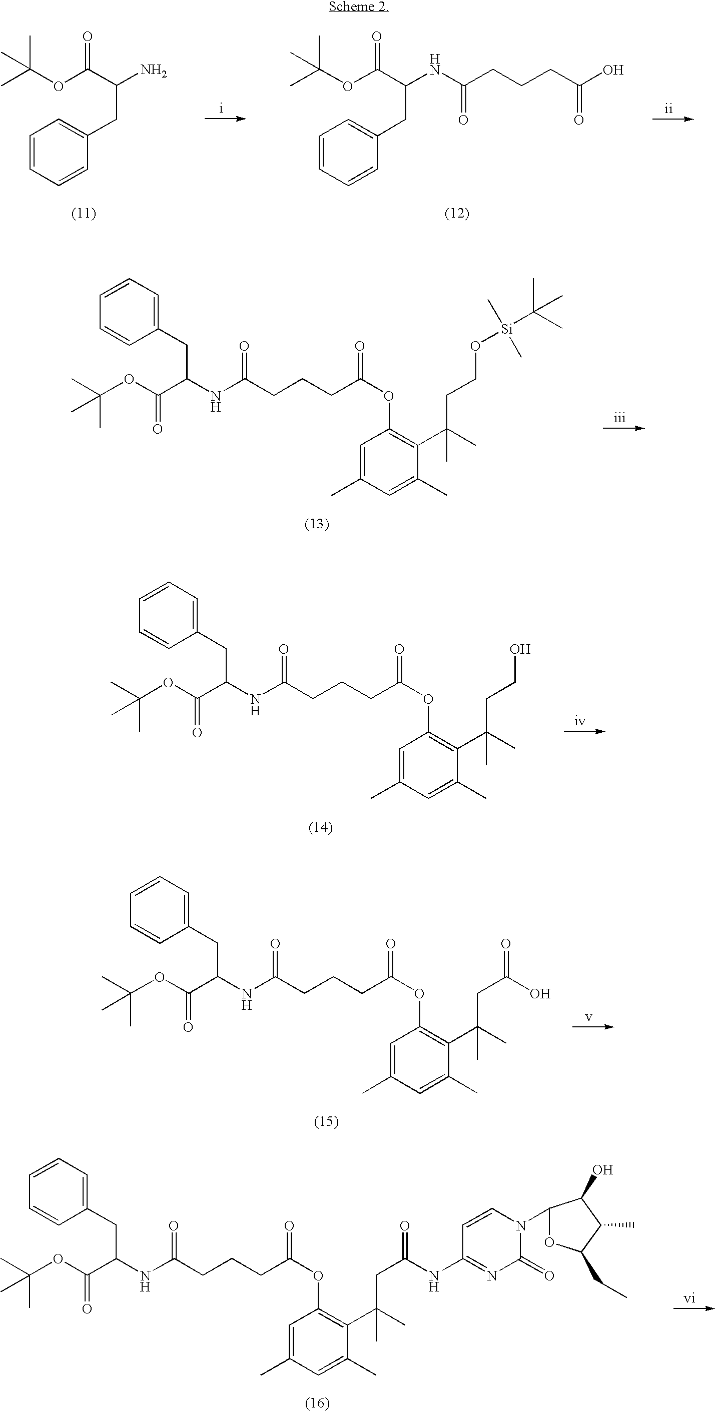

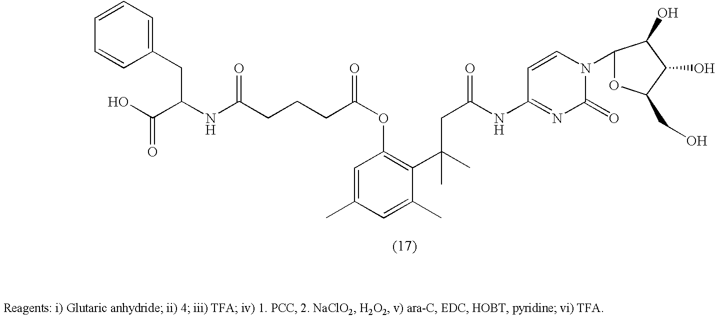

### EXAMPLE 18.1

### Synthesis of 4-(1-tert-Butoxycarbonyl-2-phenyl-ethylcarbamoyl)-butyric acid (12)

Prepared according to Dutta et al (Dutta, A. S., Gormley, J. J., McLachlan, P. F., Major, J. S. J. Med. Chem. 1990, 33, 2560-2568) for the corresponding methyl ester by reacting glutaric anhydride (1.92 g, 10 mmol), phenylalanine tert-butyl ester (1.61 g, 5 mmol), and triethylamine (3.05 g, 25 mmol) in DMF (75 ml). Yield: 2.6 g.

### EXAMPLE 18.2

### Synthesis of 1-O-(tert-Butyldimethylsilyl)-3-(2′-(4-(1-tert-butoxycarbonyl-2-phenyl-ethylcarbamoyl)-butyric acid)-4′,6′-dimethylphenyl)-3,3-dimethylpropanol (13)

Prepared by reacting EDC.HCl (1.92 g, 10 mmol), 4 (1.61 g, 5 mmol), 12 (2.51 g, 7.5 mmol) and DMAP (3.05 g, 25 mmol) in anhydrous CH₂Cl₂ (75 ml) as described for 5a. Yield: 2.6 g (90%).

¹H NMR (CDCl₃) δ 7.26-7.17 (m, 5H), 6.72 (s, 1H), 6.55 (s, 1H), 6.03 (d, 1H), 4.79 (q, 1H), 3.45 (t, 7.5 Hz, 2H), 3.10 (d, 2H), 2.57 (t, 2H), 2.55 (s, 3H), 2.29 (t, 2H), 2.23 (s, 3H), 2.04 (m, 4H), 1.46 (s, 6H), 1.42 (s, 9H), 0.86 (s, 9H), 0.00 (s, 6H).

### EXAMPLE 18.3

### Synthesis of 3-(2′-(4-(1-tert-Butoxycarbonyl-2-phenyl-ethylcarbamoyl)-butyric acid)-4′,6′-dimethylphenyl)-3,3-dimethylpropanol (14)

Prepared by reacting 13 (1.2 g, 3.75 mmol), THF (5 ml), H₂O (5 ml) and HOAc (15 ml) as described for 6a.

### EXAMPLE 18.4

### Synthesis of 3-(2′-(4-(1-tert-Butoxycarbonyl-2-phenyl-ethylcarbamoyl)-butyric acid)-3,3-dimethylpropionic acid (15)

A solution of 14 (1.97 g, 3.75 mmol) in anhydrous CH₂Cl₂ (50 ml) was slowly added to a suspension of PCC (2.33 g, 10.8 mmol) of anhydrous CH₂Cl₂ (100 ml) and stirred overnight at room temperature. The reaction mixture was evaporated in vacuo and the residue was dissolved in CH₂Cl₂ (30 ml) followed by filtration through a plug of silica (5 cm). The silica gel was flushed with diethyl ether 5 times. The filtrate was evaporated to give the a viscous oil. The oil was dissolved CH₃CN (9 ml) and added to a solution of NaH₂PO₄ (0.31 g, 2.25 mmol) in H₂O (4 ml), followed by slow addition of 80% sodium chlorite (0.85 g, 9.38 mmol) in H₂O (12 ml) at 0° C. The reaction mixture was stirred for 1 h at 0° C. and then allowed to reach room temperature. Sodium sulfite (0.47 g, 3.75 mmol) was added to the reaction to decompose HOCl and H₂O₂. The pH was adjusted to 2 using 1M aqueous HCl, followed by extraction with ethyl acetate. The organic phase washed with brine and dried over anhydrous MgSO₄. The solvent was removed in vacuo and the residue was purified by flash chromatography (10-20% MeOH in CH₂Cl₂) to give the product. Yield: 1.08 g.

¹H NMR (CDCl₃) δ 8.35 (s, broad, 1H), 7.34-7.16 (m, 5H), 6.84 (d, 1.7 Hz, 1H), 6.60 (d, 1.7 Hz, 1H), 6.38 (d, 8.1 Hz, 1H), 4.81 (q, Hz 6.3-7.8, 1H), 3.13 (d, 8.4 Hz, 2H), 2.84 (s, 2H), 2.60 (t, 7.2 Hz, 2H), 2.58 (s, 3H), 2.34 (t, 6.9 Hz, 2H), 2.25 (s, 3H), 1.60 (s, 6H), 1.44 (s, 9H).

### EXAMPLE 18.5

Synthesis of Cytarabin Derivative (16).

Prepared by reacting 15 (1.08 g, 2 mmol), ara-C (1.95 g, 8 mmol), HOBT (1.08 g, 8 mmol) and EDC.HCl (3.07 g, 16 mmol) in anhydrous pyridine (50 ml) as described for 8a. Yield: 0.5 g.

¹H NMR (CDCl₃) δ 9.25 (s, 1H), 8.63 (s, 1H), 8.12 (d, 7.5 Hz, 1H), 7.45-7.19 (m, 7H), 6.83 (d, 1H), 6.75 (d, 1.4 Hz, 1H), 6.54 (s, 1H), 6.06 (d, 3.0 Hz, 1H), 4.82 (q, 1H), 4.49 (s, 1H), 4.33 (s, 1H), 4.10 (s, 1H), 3.90-3.70 (m, 2H), 3.09 (d, 6.8 Hz, 2H), 2.92-2.77 (m, 2H), 2.60 (t, 2H), 2.50 (s, 3H), 2.32 (t, 2H), 2.15 (s, 3H), 2.01-1.84 (m, 2H), 1.70 (s, 3H), 1.65 (s, 3H), 1.37 (s, 9H);

¹³C NMR δ 173.04, 172.90, 172.18, 171.93, 162.65, 156.44, 149.72, 138.53, 136.72, 133.66, 129.83, 128.78, 127.33, 88.61, 86.00, 82.86, 54.32, 39.53, 38.33, 35.19, 34.15, 32.23, 31.96, 28.33, 25.92, 20.81, 20.63.

### EXAMPLE 18.6

### Synthesis of HO-Phe-OC(CH₂)₃CO-TML-ara-C (17)

TFA (10 ml) was added to a solution of 15 (0.29 g, mmol) in CH₂Cl₂ (10 ml) and the mixture was stirred for 2 h. The solvent was evaporated in vacuo and diethyl ether was added to precipitate the product as the TFA-salt. Yield: 0.36 g.

¹H NMR (CDCl₃) δ 10.75 (s, 1H), 8.20 (s, 1H), 7.90 (d, 7.5 Hz, 1H), 7.42-7.15 (m, 6H), 6.81 (s, 1H), 6.56 (s, 2H), 6.10 (d, 1H), 4.90 (s, 2H), 4.43 (d, 2H), 4.32 (s, 1H), 4.10 (d, 1H), 3.80 (m, 2H), 3.25 (d, 6.8 Hz, 1H), 2.99-2.75 (m, 3H), 2.52 (d, 3H), 2.31 (s, 2H), 2.20 (s, 3H), 1.97 (s, 2H), 1.55 (s, 3H), 1.40 (s, 3H);

¹³C NMR δ 176.20, 173.86, 172.78, 172.04, 149.42, 138.71, 136.73, 136.61, 133.72, 132.94, 129.54, 129.04, 127.46, 123.23, 66.29, 53.91, 39.41, 38.10, 35.20, 33.59, 32.30, 31.58, 25.85, 21.35, 20.62, 15.68.

### EXAMPLE 19

### Synthesis of ara-C 5′-O-(1-benzyl)phosphate (19)

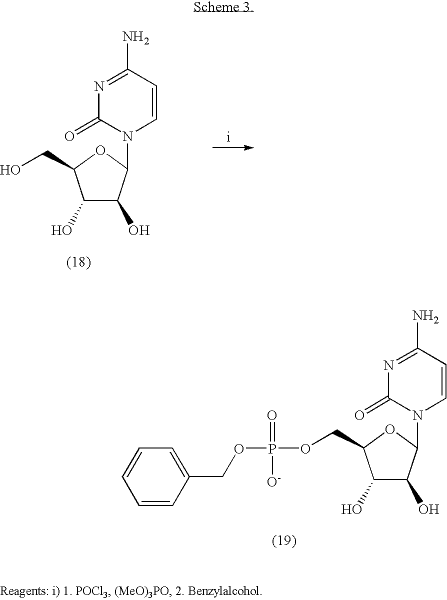

The synthesis was based on a work published by Hong et al. Hong, C. I., Kirisits, A. J., Nechaev, A., Buchheit, D. J., West, C. R. J. Med. Chem. 1985, 28, 171-177.

POCl₃ (0.5 ml) was added to a mixture of ara-C (0.73 g, 3 mmol) and trimethyl phosphate (15 ml) at −10° C. The reaction mixture was stirred at 0° C. for 4 h before benzylalcohol (15 mmol) was added. After 48 h at 4° C. the reaction mixture was poured into H₂O (250 ml) containing NaHCO₃ (1 g). The reaction was evaporated in vacuo and dissolved in MeOH (5 ml). Insoluble material was filtered and the filtrate was added diethyl ether to precipitate the product as white crystalline material.

¹H NMR (CDCl₃) δ 9.40-9.09 (d, 1H), 7.90 (d, 7.1 Hz, 1H), 7.30-7.15 (m, 5H), 6.05 (d, 3.0 Hz, 1H), 4.35-3.64 (m, 7H), 3.66 (d, 11.0 Hz, 1H), 3.43 (s, 2H), 3.40 (s, 2H), 2.84 (t, 2H);

¹³C NMR δ 162.03, 150.79, 144.96, 138.86, 129.26, 128.56, 126.45, 93.18, 87.05, 84.35, 76.19, 74.87, 65.67, 64.74, 54.40, 52.28, 36.97.

### EXAMPLE 20

### 2′,3′-dideoxy-5′-hydrogen hexadecanedioate inosine

A mixture of didanosine (300 mg), hexadecanedioic acid (1.26 g), dimethylaminopyridine (80 mg), N-(3-dimethylaminopropyl)-N′-ethyl-carbodiimide hydrochloride (300 mg) in THF (50 ml) was stirred overnight at ambient temperature. The mixture was evaporated and the title compound was isolated by flash chromatography (silica, methanol:CH₂Cl₂=2:8) plus 2% NH₃ solution (25%) as a white powder.

Yield: 323 mg (50.5%)

NMR (DMSO-d₆): 1.22-2.50 (m), 4.14-4.25 (m), 6.24 (t), 8.07(s), 8.24 (s)

MS: 505 (M+H)

### EXAMPLE 21

### 2′,3′-dideoxy-5′-hydrogen hexanedioate inosine

This compound was prepared as in Example 21 using hexanedioic acid.

Yield: 28 mg

NMR confirmed the structure

MS: 387 (M+Na)

### EXAMPLE 22

### 2′,3′-dideoxy-5′-hydrogen octanedioate inosine

The compound was prepared as in Example 21 using octanedioic acid.

Yield: 26 mg

NMR confirmed the structure

MS: 393 (M+H)

### EXAMPLE 23

### 2′,3′-dideoxy-5′-hydrogen dodecanedioate inosine

The compound was prepared as in Example 21 using dicanedioic acid.

Yield: 335 mg

NMR confirmed the structure

MS: 419 (M+H)

### EXAMPLE 24

### 5′-Boc,-2′3′-dideoxyinosine

2′,3′-dideoxyinosine (1 g, 4.24 mmol), 1,8-Diaza-7-bicyclo[5.4.0]undecene (1.29 g, 8.47 mmol), 4-dimethylaminopyridine (small catalytic amount) and DMF (10 ml) was stirred on an ice bath. Bocanhydride (1.4 g, 8.04 mmol) dissolved in DMF (10 ml) was added. The reaction mixture was stirred at room temperature over night. The reaction mixture was concentrated, 20 ml DCM added and washed with 3×20 ml NaHCO₃. The organic phase was dried with sodium sulphate, filtered, concentrated and purified by chromatography on silica using 12.5% MeOH 2% aqueous ammonia in DCM. Resulting in 611 mg white crystalline product.

46% Yield.

### EXAMPLE 25

### 5′-(2′, 3′-dideoxyinosine) mono 1,4-benzenedicarboxylic ester

2,3′dideoxyinosine (100 mg, 0.424 mmol), 1,4-benzenedicarboxylic acid (281 mg, 1.69 mmol), DMAP (20 mg, 1.64 mmol) and THF (12 ml) was stirred on an ice bath. N-(-3-Dimethylaminopropyl)-N′-ethylcarbodiimidehydrochloride (106 mg, 0.553 mmol) was added. The mixture was shaken and stirred overnight. Aqueous ammonia (1 ml), MeOH (10 ml) and DCM (10 ml) was added. The mixture was shaken, silica added, concentrated and purified by chromatography on silica using 30-40% MeOH, 5% aqueous ammonia in DCM.

The other derivates of symmetric di-acids were prepared in the same manner:

### 5′-(2′,3′-dideoxyinosine) mono 1,4-benzenedicarboxylic acid ester

20 mg, 12% yield.

### 5′-(2′,3′-dideoxyinosine) mono 1,3-benzenedicarboxylic acid ester

41 mg, 25% yield.

### 5′-(213′-dideoxyinosine) mono 1,2-benzenediacetic acid ester

70 mg, 40% yield.

### 5′-(2′,3′-dideoxyinosine) mono 1,3-benzenediacetic acid ester

77 mg, 44% yield.

### 5′-(2′,3′-dideoxyinosine) mono 1,4-benzenediacetic acid ester

73 mg, 42% yield.

### 5′-(2′,3′-dideoxyinosine) mono 1,4-benzenedipropionic acid ester

81 mg, 43% yield.

| | | % Protein |
|---|---|---|
| Example | Compound | Binding |
| 24 | 5′-Boc,-2′3′-dideoxyinosine | 8 |
| 25 | 5′-(2′,3′-dideoxyinosine) mono 1,4- | 44 |
| | benzenedicarboxylic acid ester | |
| 25 | 5′-(2′,3′-dideoxyinosine) mono 1,3- | 51 |
| | benzenedicarboxylic acid ester | |
| 25 | 5′-(2′3′-dideoxyinosine) mono 1,2- | 25 |
| | benzenediacetic acid ester | |
| 25 | 5′-(2′,3′-dideoxyinosine) mono 1,3- | 38 |
| | benzenediacetic acid ester | |
| 25 | 5′-(2′,3′-dideoxyinosine) mono 1,4- | 28 |
| | benzenediacetic acid ester | |
| 25 | 5′-(2′3′-dideoxyinosine) mono 1,4- | 63 |
| | benzenedipropionic acid ester | |

### EXAMPLE 26

Albumin Binding of Didanosine and Didanosine Prodrugs

The method for determination of protein binding is described in Example 7

| | % Albumin |
|---|---|
| Compound | binding |
| Didanosine (2′,3′-dideoxy-inosine) | less |
| | than 5 |
| 2′,3′-dideoxy-5′-hydrogen | 10 |
| hexanedioate-inosine (Example 22) | |
| 2′,3′-dideoxy-5′-hydrogen | 40 |
| octanedioate-inosine (Example 23) | |
| 2′,3′-dideoxy-5′-hydrogen | 94 |
| dodecanedioate-inosine (Example 24) | |
| 2′,3′-dideoxy-5′-hydrogen | 100 |
| hexadecanedioate-inosine (Example 21) | |

### EXAMPLE 27

### Albumin Binding of 6-Mercaptopurine and 6-Mercaptopurine Prodrugs

The method for determination of protein binding is described in Example 7

| | % Albumin |
|---|---|
| Compound | binding |
| 6-Mercaptopurine | 10 |
| 6-(1H-purin-6-ylthio)-hexanoic | 72 |
| acid methyl ester (Example 9) | |
| 12-(1H-purin-6-ylthio)-dodecanoic | 100 |
| acid (Example 11) | |
| 12-(1H-purin-6-ylthio)-dodecanoic | insoluble |
| acid methyl ester (Example 10) | |

### EXAMPLE 28

### 1-(5-O-Azelayl-β-D-arabinofuranosyl)-cytosine

A mixture of the methyl ester of the title compound (220 mg, 0.51 mmol) and potassium hydroxide (34 mg, 0.51 mmol) in a mixture of ethanol and water (1:1, 4 ml) was stirred at ambient temperature for 12 hours. The reaction mixture was evaporated. LCMS confirmed that the title compound was formed.

### EXAMPLE 29

### Intermediate for Protection of Monoester Derivatives of Various Drugs

Mono Protection of Dicarboxylic Acids

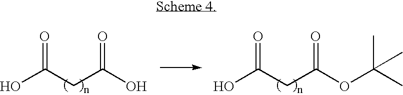

Mono protection of dicarboxylic acids according to Scheme 4 was prepared using the method developed by Ogawa, Y., Kodaka, M., Okuno, H. Chemistry and Physics of Lipids 2002, 119, 51-68.

1. A prodrug compound comprising a therapeutically effective moiety coupled via a metabolically cleavable bond to a blood protein binding moiety.

2. The prodrug compound as claimed in claim 1, wherein said compound is water-soluble.

3. The prodrug compound as claimed in claim 1, wherein said metabolically cleavable bond is an oxidatively cleavable bond.

4. The prodrug compound as claimed in claim 1, wherein said metabolically cleavable bond is an ester bond.

5. The prodrug as claimed in claim 1, wherein said protein binding moiety is an acid moiety.

6. The prodrug compound as claimed in claim 5, wherein said protein binding moiety is a carboxylic acid moiety.

7. The prodrug compound as claimed in claim 1, wherein said protein binding moiety is an esterified acid moiety.

8. The prodrug compound as claimed in claim 1, wherein said metabolically cleavable bond is distanced from the protein binding moiety by a group, —CH₂—CH₂—R—, where the —CH₂—CH₂— component is attached to or by the metabolically cleavable bonding and is a hydrocarbyl linker containing up to 30 carbon atoms.

9. The prodrug compound as claimed in claim 1, wherein said therapeutically effective moiety is selected from the group consisting of metronidazole, 6-mercaptopurine, 5-fluorouracil, cytarabine and didanosine.

10. The prodrug compound as claimed in claim 1, wherein said protein binding moiety is an ester-bound azelaic acid or an ester thereof.

11. A pharmaceutical composition comprising a prodrug compound as claimed in claim 1, together with at least one pharmaceutically acceptable carrier or excipient.

12. A method of treatment of a human or non-human vascularized animal subject, which method comprises parenterally administering to said subject an effective amount of a prodrug as claimed in claim 1.

13. A process for the preparation of a prodrug as claimed in claim 1, which process comprises coupling a therapeutically active drug compound (or a salt or activated derivative thereon) and a blood protein-binding agent.

14. A prodrug comprising a therapeutically effective moiety coupled via a metabolically cleavable bond to a blood protein binding moiety, wherein the therapeutically effective moiety is selected from the group consisting of a metronidazole, 6-mercaptopurine, 5-fluorouracil, cytarabine and didanosine and the protein binding moiety is an ester-bound azelaic acid, optionally with its second carboxyl group ester-protected.

15. A pharmaceutical composition comprising the prodrug compound as claimed in claim 11, which is preferably a solution for injection.
